(19) United States
(12) Patent Application Publication
(10) Pub. No.: US 2026/0314971 A1
(43) Pub. Date: Oct. 15, 2026

(54) SYSTEMS AND METHODS FOR DETERMINING A VOLUME OF URINE IN A BLADDER

(71) Applicant: Medtronic, Inc., Minneapolis, MN (US)

(72) Inventor: Andrew T. Fried, St. Paul, MN (US)

(21) Appl. No.: 19/630,544

(22) Filed: Mar. 27, 2026

Related U.S. Application Data
(60) Provisional application No. 63/779,536, filed on Mar. 28, 2025.

(51) Int. Cl. A61B 5/20; A61B 5/00; A61B 5/05

(52) U.S. Cl. CPC A61B 5/204; A61B 5/05; A61B 5/7267; A61B 5/7282

(57) ABSTRACT

One aspect of the present disclosure relates generally to a method for determining a volume of fluid in a bladder. The method can include determining a calibrated bladder volume when the bladder is substantially empty. Furthermore, the method can include determining a volume of fluid in the bladder by generating a second random noise signal when the bladder contains the fluid, transmitting the second random noise signal using the transducer positioned within the bladder, receiving a second return signal using the receiver positioned within the bladder, determining a second resonant frequency of the second return signal, and determining a volume of fluid in the bladder by comparing the first resonant frequency and the second resonant frequency. Further yet, the method can include generating, for output to a user, the volume of fluid in the bladder.

### CROSS-REFERENCE TO RELATED APPLICATION

This application claims the benefit of and priority to U.S. Provisional Patent Application No. 63/779,536 filed Mar. 28, 2025, the entire disclosure of which is incorporated by reference herein.

### FIELD

In certain embodiments, the present technology is generally related to determining a volume of urine in a bladder of a patient. In some embodiments, the present technology is generally related to determining a volume of urine or fecal waste excreted from a patient or during an elimination event.

### BACKGROUND

Measuring the volume of fluid in the bladder is an important part of diagnosing and monitoring urinary conditions. The bladder's capacity can vary depending on factors like age, gender, and overall health. Accurate measurement of bladder volume helps identify issues such as urinary retention, incontinence, or overactive bladder, and is essential for determining the severity of these conditions. It is desirable to monitor a volume of fluid in a bladder during post-surgical recovery and while managing patients with neurological disorders that affect bladder control.

### SUMMARY

In certain embodiments, the present disclosure includes a system for determining a volume of fluid in a bladder comprising a transducer positioned within the bladder and configured to emit a random noise signal, a receiver positioned within the bladder and configured to receive a response to the random noise signal, and processing circuitry. The processing circuitry may be configured to: generate a first random noise signal when the bladder is substantially empty using a signal transducer, transmit the first broadband random noise signal using a transducer positioned in the bladder, receive a first return signal using a receiver positioned in the bladder, determine a first resonant frequency of the return signal, and determining, based on the resonant frequency of the return signal, the calibrated bladder volume. The processing circuitry may be further configured to determine the volume of fluid in the bladder by: generate a second random noise signal when the bladder contains the fluid, transmit the second random noise signal using the transducer positioned within the bladder, receive a second return signal using the receiver positioned within the bladder, determine a second resonant frequency of the second return signal, and determine a volume of fluid in the bladder by comparing the first resonant frequency and the second resonant frequency. The processing circuitry may be further configured to generate, for output to a user, the volume of fluid in the bladder.

In certain embodiments, the present disclosure includes a system for determining a volume of material excreted during an elimination event comprising: a receiver configured to receive a noise signal generated from a patient during the elimination event; and a smart device comprising: a processor; and a computer-readable medium storing instructions which, when executed by the processor, cause the processor to: calibrate the smart device to a toilet used by a patient to estimate an initial volume of fluid within the toilet prior to an elimination; receive the noise signal generated from the patient during elimination; identify one or more characteristics of the noise signal comprising at least one of a noise frequency, a noise amplitude, a duration of sound events, and other acoustic properties; process the one or more characteristics of the noise signal using a machine learning model that is trained to correlate the one or more characteristics with a known volume of fluid in the toilet to estimate a total volume of fluid after elimination; estimate a change in volume by subtracting the initial volume of fluid in the toilet from the total volume of fluid after elimination; and generate, for output to a user, the change in volume.

In certain embodiments, the present disclosure includes a method for determining a volume of fluid in a bladder comprising: determining a calibrated bladder volume by: generating a first random noise signal when the bladder is substantially empty using a signal transducer, transmitting the first broadband random noise signal using a transducer positioned in the bladder, receiving a first return signal using a receiver positioned in the bladder, determining a first resonant frequency of the return signal, and determining, based on the resonant frequency of the return signal, the calibrated bladder volume. The method may further include determining the volume of fluid in the bladder by: generating a second random noise signal when the bladder contains the fluid, transmitting the second random noise signal using the transducer positioned within the bladder, receiving a second return signal using the receiver positioned within the bladder, determining a second resonant frequency of the second return signal, and determining a volume of fluid in the bladder by comparing the first resonant frequency and the second resonant frequency. The method may further include generating, for output to a user, the volume of fluid in the bladder.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic diagram of a system for determining a volume of fluid in a bladder, in accordance with one or more embodiments of the present disclosure.

FIG. 2 is a schematic diagram of a system for determining a volume of material excreted during an elimination event, in accordance with one or more embodiments of the present disclosure.

FIG. 3 is a block diagram illustrating example system inputs for the systems of FIGS. 1-2, in accordance with one or more embodiments of the present disclosure.

FIG. 4 is a block diagram illustrating example system outputs for the systems of FIGS. 1-2, in accordance with one or more embodiments of the present disclosure.

FIG. 5 is a flow chart diagram of a method for determining a volume of fluid in a bladder, in accordance with one or more embodiments of the present disclosure.

FIG. 6 is a flow chart diagram of a method for calibrating a volume measurement system to find a first resonant frequency of FIG. 5, in accordance with one or more embodiments of the present disclosure.

FIG. 7 is a flow chart diagram of a method for determining a second resonant frequency of a bladder with fluid of FIG. 5, in accordance with one or more embodiments of the present disclosure.

FIG. 8 is a flow chart diagram of a method for determining a volume of material excreted during an elimination event, in accordance with one or more embodiments of the present disclosure.

FIG. 9 is a flow chart diagram of a method of monitoring excrement with a smart device, in accordance with one or more embodiments of the present disclosure.

FIG. 10 is a flow chart diagram of another method for monitoring excrement with a smart device, in accordance with one or more embodiments of the present disclosure.

FIG. 11 is a flow diagram of a method for monitoring excrement with a user, a smart device, a sensor device, and a cloud system, in accordance with one or more embodiments of the present disclosure.

FIG. 12 is graph of amplitude vs. time (including a Fast Fourier Transform illustrating amplitude vs. frequency in the graph below) of an elimination event including background noise, in accordance with one or more embodiments of the present disclosure.

FIG. 13A is graph of amplitude vs. time (including a Fast Fourier Transform illustrating amplitude vs. frequency in the graph below) of an elimination event with a standing male user, in accordance with one or more embodiments of the present disclosure.

FIG. 13B is a second amplitude vs. time (including a Fast Fourier Transform illustrating amplitude vs. frequency in the graph below) of an elimination event with a standing male user, in accordance with one or more embodiments of the present disclosure.

FIG. 14 is a graph showing two events where sounds associated with filling a container were recorded within an empty space and a blue shift from was observed from approximately 300-1000 Hz and from the time at about the −90 db level, in accordance with one or more embodiments of the present disclosure.

### DETAILED DESCRIPTION

In certain embodiments, the present technology is generally related to determining a volume of urine in a bladder of a patient. In some embodiments, the present technology is generally related to determining a volume of urine or fecal waste excreted from a patient or during an elimination event.

Measuring the volume of urine in the bladder or the volume of fecal/urine waste excreted during an elimination event through non-invasive methods is desirable for both medical and practical reasons. Non-invasive techniques eliminate the need for uncomfortable or risky procedures, promoting patient comfort and safety. Methods such as ultrasound, acoustic sensors, or advanced imaging technologies can accurately determine fluid levels or waste volume without physical contact. These approaches are particularly valuable in monitoring patients with conditions such as urinary retention, incontinence, or gastrointestinal disorders, as they offer real-time insights while minimizing the risk of infection or injury. Additionally, non-invasive methods are more suitable for continuous monitoring in settings such as hospitals, long-term care, or even at home, enhancing patient convenience and compliance. Ultimately, these techniques provide accurate, safe, and efficient ways to track bodily functions, facilitating better diagnosis, treatment, and overall care.

Determining the volume of urine in the bladder and the amount of urine or feces eliminated into a toilet is desirable for monitoring pelvic health, as it provides valuable insights into the function of the urinary and gastrointestinal systems. Tracking urine volume helps detect issues such as urinary retention, incontinence, or overactive bladder, while also assessing kidney function and hydration levels. Monitoring fecal elimination can reveal signs of constipation, irregular bowel movements, or other gastrointestinal concerns. By regularly measuring these volumes, healthcare providers can better understand a patient's condition, identify early signs of potential problems, and tailor treatments to improve overall pelvic health. This information is also crucial for managing chronic conditions and guiding interventions, ultimately leading to better outcomes and quality of life for patients.

A “fluid” as described herein generally refers to any human bodily fluid, including, by non-limiting example, urine, fecal matter, or both.

An “elimination event” as described herein generally refers to an event where a person urinates, defecates, or both.

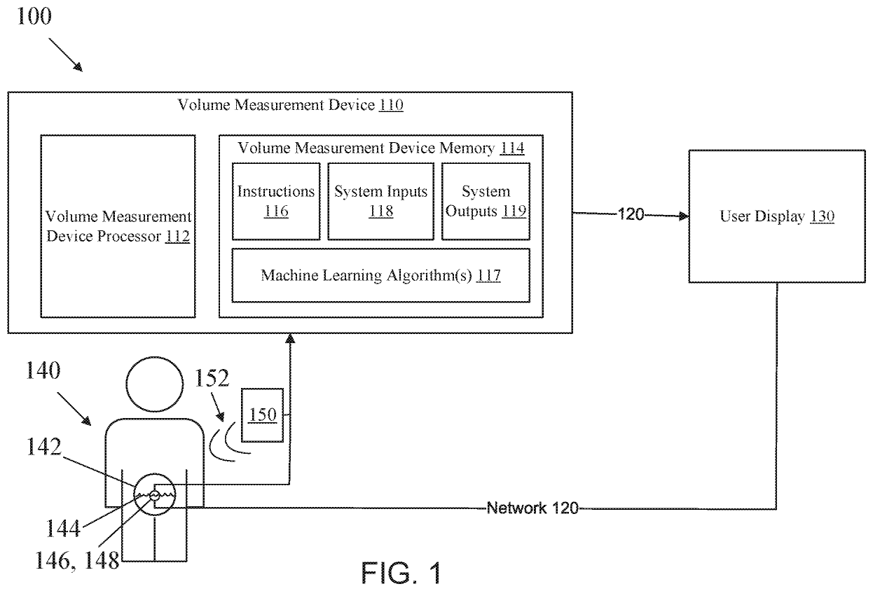

FIG. 1 is a schematic diagram of a system 100 for determining a volume of fluid in a bladder 142, in accordance with one or more embodiments of the present disclosure. The system 100 includes a volume measurement device 110, a user display 130, and one or more components connected to a patient 140 that communicate via a network 120.

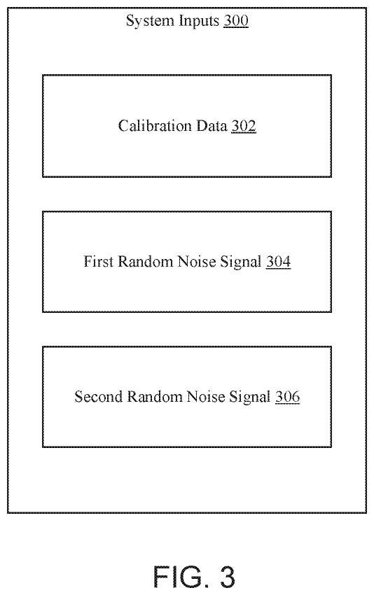

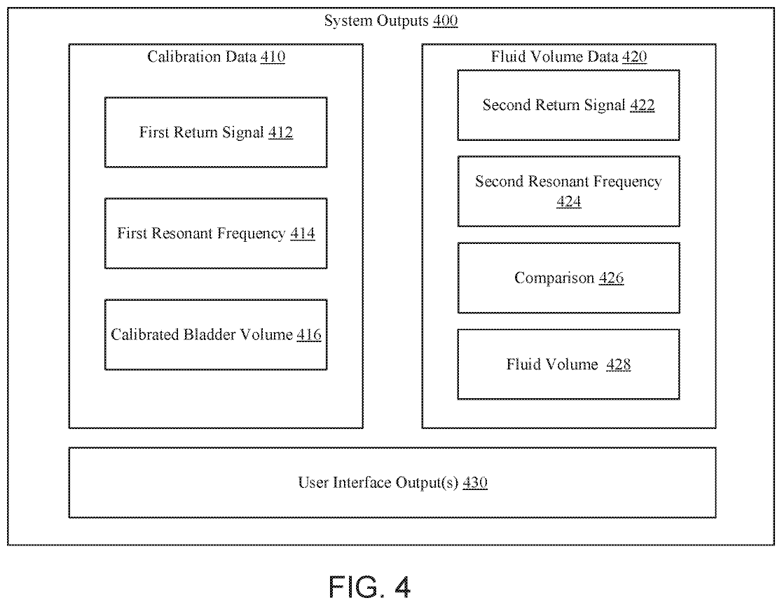

In certain embodiments, the volume measurement device 110 includes a volume measurement device processor 112 and a volume measurement device memory 114. The volume measurement device memory 114 may include a computer-readable medium storing instructions 116 which, when executed by the volume measurement device processor 112, cause the processor to execute one or more processes described herein. The volume measurement device memory 114 may include system inputs 118, system outputs 119, and one or more machine learning algorithms 117. The system inputs 118 are illustrated and described in further detail with respect to FIG. 3. The system outputs 119 are illustrated and described in further detail with respect to FIG. 4.

The one or more machine learning algorithms 117 can be stored in the volume measurement device memory 114 or externally on a server to execute one or more of the methods described herein. A machine learning algorithm 117 is a computational model that allows the system 100 to learn patterns or make predictions based on data (e.g., the system inputs 300), without being explicitly programmed for every task. The one or more machine learning algorithms 117 analyze data, recognize patterns, and improve its performance over time as it processes additional information. A primary goal of machine learning is to enable systems to generalize from data, allowing them to make accurate predictions or decisions when faced with new, unseen data.

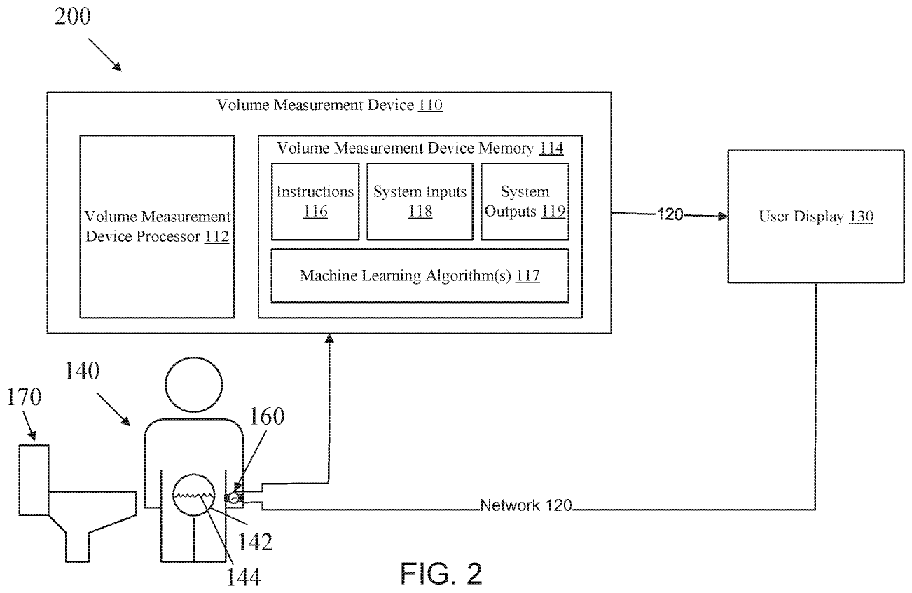

In certain embodiment, the machine learning algorithm(s) 117 can include supervised learning or unsupervised learning. In supervised learning, the one or more machine learning algorithms 117 are trained on by providing a labeled dataset (e.g., a labeled dataset corresponding to one or more patients or systems 100), meaning that the input data is paired with correct output labels (e.g., a labeled impedance measurement). The one or more machine learning algorithms 117 learn to map inputs to outputs (e.g., the system inputs 300 and the system outputs 400) by minimizing the difference between its predictions and the actual outcomes. Machine learning algorithm(s) 117 described herein may be used to generate models for predicting various acoustic parameters within one or more patients as illustrated and described herein with respect to FIGS. 2-8.

On the other hand, unsupervised learning involves training a model on data without predefined labels. The one or more machine learning algorithms 117 seek to identify hidden patterns or structures in the data, such as grouping similar data points into clusters (clustering) or reducing the dimensionality of complex data sets (dimensionality reduction). In certain embodiments, unsupervised learning is used to perform one or more of the methods discussed herein.

Embodiments within the scope of the present disclosure also include physical and other computer-readable media for carrying or storing computer-executable instructions 116 and/or data structures, including applications, tables, data, libraries, or other modules used to execute functions or direct selection or execution of other modules. Such computer-readable media can be any available media that can be accessed by a general purpose or special purpose computer system. Computer-readable media that store computer-executable instructions (or software instructions) are physical storage media. Computer-readable media that carry computer-executable instructions are transmission media. Thus, by way of example, and not limitation, embodiments of the present disclosure can include at least two distinctly different kinds of computer-readable media, namely physical storage media or transmission media. Combinations of physical storage media and transmission media should also be included within the scope of computer-readable media.

Both physical storage media and transmission media may be used temporarily to store or carry software instructions in the form of computer readable program code that allows performance of embodiments of the present disclosure. Physical storage media may further be used to persistently or permanently store such software instructions. Examples of physical storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored as or in software, hardware, firmware, or combinations thereof.

A network 120 or “communications network” may generally be defined as one or more data links that enable the transport of electronic data between computer systems and/or modules, engines, and/or other electronic devices. When information is transferred or provided over a communication network or another communications connection (either hardwired, wireless, or a combination of hardware or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instructions or data structures and which can be accessed by a general purpose or special purpose computer. As illustrated and described in FIG. 1, the network 120 enables transport of electronic data between the volume measurement device 110, a user display 130, and other device(s) (e.g., a transducer 146, a receiver 148, or an ultrasound device 150), which are illustrated and described in further detail below.

Further, upon reaching various computer system components, program code in the form of computer-executable instructions or data structures can be transferred automatically or manually from transmission media to physical storage media (or vice versa). For example, computer-executable instructions or data structures received over a network or data link can be buffered in memory (e.g., RAM) within a network interface module (NIC), and then eventually transferred to computer system RAM and/or to less volatile physical storage media at a computer system. Thus, it should be understood that physical storage media can be included in computer system components that also (or even primarily) utilize transmission media.

In certain examples, one or more lookup tables can be stored in the volume measurement device memory 114 or externally on a server to execute one or more of the methods described herein

The user display 130 can include a button or keypad, lights, a speaker for voice commands, a knob able to turn, a display, such as a liquid crystal display (LCD), light-emitting diode (LED), or cathode ray tube (CRT). In some examples, the display may be a touch screen. In certain embodiments, the volume measurement device 110 includes the user display 130 for interacting with a user (e.g., a patient or a healthcare provider).

In certain embodiments, the system 100 includes a transducer 146 and a receiver 148 positioned within the bladder 142 (holding a volume of liquid 144) of a patient 140. The transducer 146 is a device that converts one form of energy into another, typically used in various measurement and sensing applications. In the context of monitoring bladder volume, a transducer typically converts sound waves (e.g., ultrasound) or other forms of energy into electrical signals. In certain embodiments, the transducer 146 can include a speaker for generating a random noise signal, such as a broadband random noise signal. In certain embodiments, this broadband noise signal can include white noise, brown noise, pink noise, or combinations thereof. A transducer 146 emitting a broadband random noise signal works by generating a wide range of frequencies that interact with an object, such as the bladder 142 or another container. The bladder 142 responds to these frequencies, with certain frequencies causing stronger vibrations or reflections due to the object's resonant frequency—the frequency at which it naturally vibrates most easily.

In at least one embodiment, one or more components described herein (e.g., the transducer 146 and receiver 148) can be inserted into the bladder 142 using a flexible catheter or other means of transurethral insertion. In some embodiments, the one or more components described herein (e.g., the transducer 146 and receiver 148) can be inserted into the bladder 142 using small incision in the abdomen that allows a surgeon to place the one or more components on the outside or inside of the bladder 142. In certain embodiments where the one or more components are placed inside of the bladder 142, a small incision could be made to access the inside of the bladder 142 or the one or more components can be injected into the bladder 142.

A receiver 148 detects these reflections or vibrations, and the signal is processed to identify the dominant frequencies where the response is strongest, indicating the bladder's resonant frequencies. By analyzing shifts in the resonant frequency, often caused by changes in volume (e.g., the amount of fluid in a bladder), the system 100 can estimate the volume of fluid or waste present, making this a non-invasive method for measuring fluid volume or waste elimination.

In another example, an ultrasound transducer 150 can emits sound waves 152 that travel through the body or surrounding material. These waves reflect off surfaces like the bladder walls or waste, and the transducer detects these reflections, which are then converted into data. The transducer in this context can include components such as piezoelectric crystals or membranes that vibrate to generate or receive sound waves, and it is integral to non-invasive measurement techniques used to assess fluid or waste volume.

FIG. 2 is a schematic diagram of a system 200 for determining a volume of material excreted during an elimination event, in accordance with one or more embodiments of the present disclosure. In certain embodiments, the system 200 can include one or more components illustrated and described above with respect to FIG. 1, such as the volume measurement device 110, the network 120, and the user display 130.

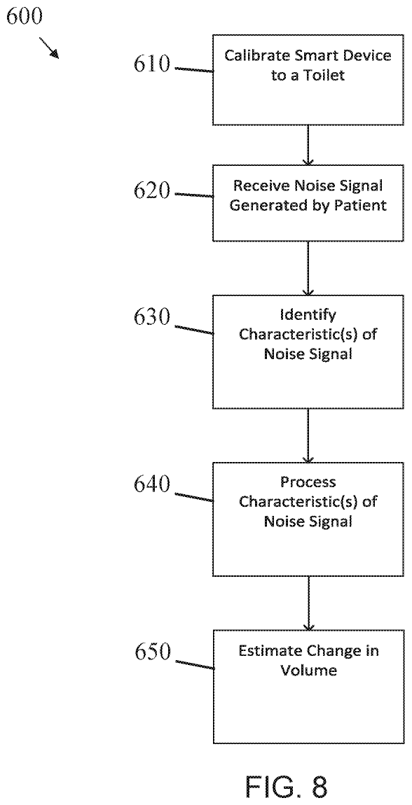

In some embodiments, the system 200 includes a smart device 160 (e.g., a smart watch 160 worn by the patient 140). A smart device 160 is an electronic gadget that connects to the internet or a network (e.g., the network 120), allowing it to interact with users and other devices through sensors, software, and data processing. Smart devices can automate tasks, learn from data, and adapt to user preferences. Examples of smart devices worn or used by a patient can include smartwatches that track health metrics, fitness trackers that monitor physical activity, smart rings that track sleep and activity, and smart clothing embedded with sensors to monitor body metrics. These devices may enhance daily life by offering real-time insights, automating tasks, and helping users monitor their health and activities. A method for determining a volume of material excreted during an elimination event into a toilet 170 using the smart device 160 is illustrated and described in further detail with respect to FIG. 8.

FIG. 3 is a block diagram illustrating example system inputs 300 for the systems 100, 200 of FIGS. 1-2, in accordance with one or more embodiments of the present disclosure. In certain embodiments, the system inputs 300 include calibration data 302, a first random noise signal 304, and a second random noise signal 306.

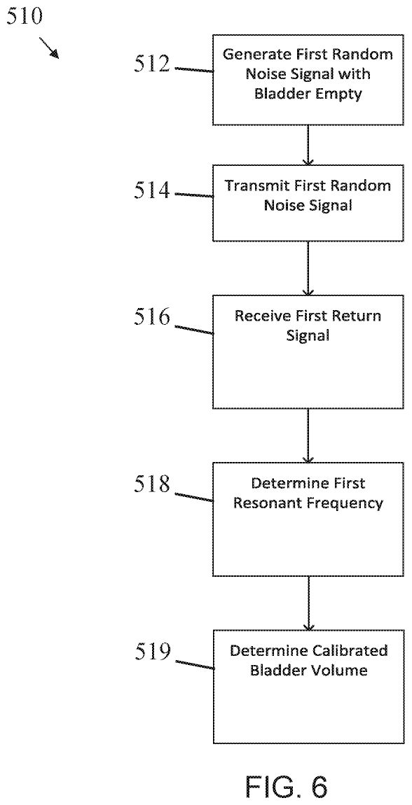

The calibration data 302 can include one or more measurements pertaining to a patient's bladder, including the volume of the patient's bladder when it is substantially empty (i.e., after the patient goes to the bathroom or when a catheter is used to drain fluid from the bladder). In at least some patients, substantially emptying a bladder means that not all urine is eliminated during an elimination event. In certain embodiments, the calibration data 302 can be stored in the volume measurement device memory 114 after performing calibration steps to measure the volume of a bladder that is substantially empty (e.g., by performing the steps that are illustrated and described in further detail with respect to FIG. 6). By storing calibration data, a calibration step need be performed only once to determine the volume of a patient's bladder and a first resonant frequency associated with the patient's bladder when it is substantially empty. In some embodiments, calibration may occur over time to determine the volume of the patient's substantially empty bladder after a plurality of urination events.

The first random noise signal 304 and the second random noise signal 306 include a broadband noise signal can include white noise, brown noise, pink noise, or combinations thereof. In certain embodiments, the first random noise signal 304 can include a range of frequencies between 20 Hz to 20,000 Hz (e.g., the audible spectrum for humans). In certain embodiments, the first random noise signal 304 and the second random noise signal 306 can include the same range of frequencies.

FIG. 4 is a block diagram illustrating example system outputs 400 for the systems 100, 200 of FIGS. 1-2, in accordance with one or more embodiments of the present disclosure. The example system outputs 400 may include calibration data 410, fluid volume data 420, and other user interface outputs 430.

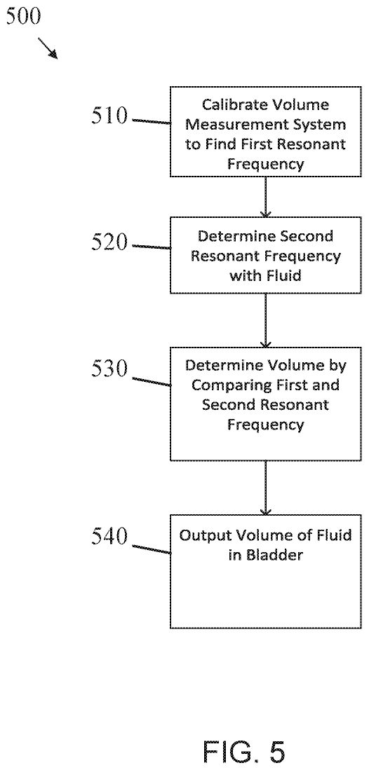

In certain embodiments, the calibration data 410 includes one or more measurements taken to calibrate the system 100, 200. This may include, by non-limiting example, generating a first random noise signal 304 when the bladder is substantially empty using a signal transducer, transmitting the first random noise signal 304 using a transducer 146 positioned in the bladder 142, receiving a first return signal 412 using a receiver 148 positioned in the bladder 142, determining a first resonant frequency 414 of the first return signal 412, and determining, based on the first resonant frequency 414 of the first return signal 412, the calibrated bladder volume 416. A method for calibrating the system 100, 200 is illustrated and described in further detail with respect to FIGS. 5-8.

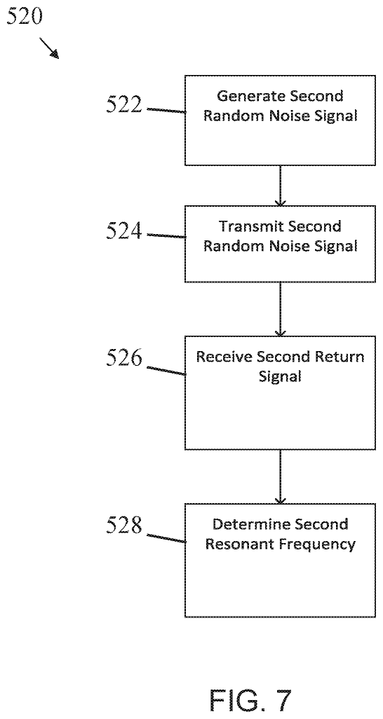

In one or more embodiments, the fluid volume data 420 includes a second return signal 422, a second resonant frequency 424, a comparison 426 between the first resonant frequency 414 and the second resonant frequency 424, and a fluid volume 428. A method for calculating the fluid volume data 420 is illustrated and described in further detail with respect to FIGS. 5 and 7.

The user interface outputs 430 may include one or more of the system inputs 300 or the system outputs 400 to display to a user on the user display 130. Furthermore, the user interface outputs 430 may include alerts or recommendations generated by the system 100, 200 based on the system outputs 400. The alerts or recommendations can include, but are not limited to, alerting a patient or healthcare provider of the status of the volume of urine in a patient's bladder, recommending a patient eliminate urine or feces at a certain time, recommending one or more additional measurements be taken by the system 100, 200, or recommending other medical interventions be provided to the patient based on the system outputs 400 or an output generated by the one or more machine learning algorithms 117.

FIG. 5 is a flow chart diagram of a method 500 for determining a volume of fluid in a bladder, in accordance with one or more embodiments of the present disclosure.

The method 500 includes a step 510 of calibrating a volume measurement system 100, 200 to find a first resonant frequency. Step 510 is illustrated and described in further detail with respect to FIG. 6.

The method 500 includes a step 520 of determining a second resonant frequency with fluid present in the bladder of a patient. Step 520 is illustrated and described in further detail with respect to FIG. 7.

The method includes a step 530 of determining a volume of fluid in the bladder by comparing the first resonant frequency 414 (e.g., a resonant frequency measured when the bladder is substantially empty) and the second resonant frequency 424 (e.g., a resonant frequency measured at a time after calibration). To determine the volume of fluid in a bladder after obtaining the first resonant frequency 414 and the second resonant frequency 424, the resonant frequencies are compared to identify a frequency shift between the two resonant frequencies measured. The resonant frequency of a bladder depends on the properties of the medium inside it, including its volume and density. When fluid is present, the added mass and altered acoustic properties cause a measurable decrease in the resonant frequency compared to the empty bladder. By using a predetermined calibration curve or mathematical model (e.g., a mathematical model created by the one or more machine learning algorithms 117) that correlates frequency shifts to bladder volume, the difference between the first (empty) and second (fluid-filled) resonant frequencies can be used to calculate the volume of fluid present. This method relies on accurate measurements of the resonant frequencies and an established relationship between frequency shifts and fluid volume.

The method 500 includes a step 540 of outputting a volume of fluid in the bladder. In certain embodiments, this may include providing the fluid volume 428 and one or more user interface outputs 430 to a user by transmitting this information to a user display 130 via the network 120.

FIG. 6 is a flow chart diagram of a method 510 for calibrating a volume measurement system to find the first resonant frequency of FIG. 5, in accordance with one or more embodiments of the present disclosure.

The method 510 includes a step 512 of generating a first random noise signal 304 when the bladder 142 is substantially empty. As illustrated and described above in further detail with respect to FIGS. 1-5, the first random noise signal 304 can include a broadband range of noise signals including white noise, brown noise, pink noise, and other frequencies.

The method 510 includes a step 514 of transmitting the first random noise signal 304 using the transmitter 146. As illustrated and described above with reference to FIGS. 1-5, the transducer 146 can include a plurality of different transducer types that are suitable for transmitting a signal within the human body, such as an ultrasound transducer 150, a bone conduction transducer, an acoustic transducer, an electromagnetic transducer, a hydrophone transducer, a vibroacoustic transducer, or other means for transducing an acoustic signal.

The method 510 includes a step 516 of receiving the first return signal 304 using a receiver 148. As illustrated and described above with reference to FIGS. 1-5, the receiver 148 can include, by non-limiting example, one or more different types of receivers for receiving a signal within the human body (e.g., a microphone, an accelerometer, a hydrophone, an ultrasound receiver, acoustic emission sensors, and micro-electro-mechanical systems (MEMS) microphones).

The method 510 includes a step 518 of determining a first resonant frequency 414 from the first return signal 412. A resonant frequency can be determined from a return signal picked up by a receiver by analyzing the frequency response of the system. When an object or system is excited by an external sound source, it vibrates most strongly at its natural resonant frequency, producing a distinct peak in the return signal. The microphone captures this return signal, which is then processed by using one or more techniques to analyze its frequency components (e.g., Fourier Transform). The frequency with the highest amplitude in the spectrum corresponds to the resonant frequency, as it represents the strongest response of the system to the excitation. In some embodiments, the one or more machine learning algorithms 117 can be trained to identify the resonant frequency of a system that is unique to a patient's bladder or it can predict a resonant frequency of a patient's bladder based on patient demographic data.

The method 510 includes a step 519 of determining a calibrated bladder volume. To estimate the volume of a bladder using the first resonant frequency, you can rely on the relationship between the resonant frequency of a fluid-filled cavity and its physical dimensions. When a bladder is excited acoustically, it resonates at specific frequencies determined by its size, shape, and the properties of the fluid inside. Once the resonant frequency is identified, mathematical models or empirical formulas can be used to relate the frequency to the volume of the bladder. For a roughly spherical bladder, the resonant frequency is inversely proportional to the square root of the volume. Simple calculations can be made when assuming the bladder's shape is roughly spherical, whereas more complex calculations can be made to find the exact dimensions of the bladder while accounting for other material properties like wall elasticity of the bladder.

FIG. 7 is a flow chart diagram of a method 520 for determining a second resonant frequency 424 of a bladder 142 with fluid of FIG. 5, in accordance with one or more embodiments of the present disclosure.

The method 520 includes one or more steps that may be performed similarly to the steps illustrated and described above with respect to FIG. 6. In certain embodiments, the method includes steps of generating a second random noise signal 522, transmitting the second random noise signal using the transducer 524, receiving a second return signal 526, and determining a second resonant frequency with fluid present in the bladder 528.

FIG. 8 is a flow chart diagram of a method 600 for determining a volume of material excreted during an elimination event, in accordance with one or more embodiments of the present disclosure.

The method 600 includes a step 610 of calibrating a smart device to a toilet. Calibrating a smart device (e.g., a smart watch) to estimate the volume of urine or feces eliminated into a toilet based on acoustic signals may include a plurality of steps. First, the specific toilet's acoustic properties must be characterized, as different toilets have unique shapes, materials, and resonances. This can be done by introducing controlled volumes of water or simulated waste into the toilet and recording the corresponding acoustic signals using the device. These signals would then be analyzed to identify patterns or frequencies that correlate with specific volume changes. The one or more machine learning algorithms 117 could be trained on this data to map acoustic features to volume estimates accurately. Additionally, environmental factors such as background noise or variations in user behavior (e.g., distance from the device) should be accounted for by testing in real-world conditions and refining the algorithm accordingly. Periodic recalibration may be necessary to maintain accuracy, especially if the toilet environment or device placement changes.

The method 600 includes a step 620 of receiving a noise signal generated by the patient. The smart device 160 may include a receiver or communicate with a receiver that is positioned externally from the smart device 160 to receive the noise signal generated by the patient during elimination into a toilet. Furthermore, if the noise signal generated by the patient does not have a great enough amplitude, additional random noise can be introduced using the smart device 160 to find an acoustic resonance of the volume of fluid in the toilet after elimination is complete.

In certain embodiments, a user may be prompted to provide consent to record prior to receiving a noise signal generated by the patient. This may include, by non-limiting example, transmitting a request to the user on a digital display and receiving a response from the patient indicating whether the patient provides consent to record the noise signal generated by the patient.

In at least one embodiment, the system may instruct a user to hold the smart device 160 over an edge of the toilet to allow the smart device 160 to emit a sound wave and listen to a response to the sound wave. This may be desirable if noise signals generated by the patient are unable to be measured by the smart device.

In other embodiments, the system may include a smart device 160 that is connected to a wearable device (e.g., a smart patch including an adhesive configured to be stuck to the patient). The wearable device may be positioned on the posterior side of the patient's upper leg where it faces downward into the toilet to collect sound signals resonating within the toilet. The wearable device may communicate with the smart device 160 through any reasonable means as described above (e.g., over a network such as Bluetooth, Wi-Fi, or the like).

The method 600 includes a step 630 of identifying one or more characteristics of the noise signal. The one or more characteristics of the noise signal may include, but are not limited to, at least one of a noise frequency, a noise amplitude, a duration of the sound event, and other acoustic properties such as environmental conditions (e.g., temperature, humidity, and air pressure) material properties of the toilet, a geometry of the toilet, and a resonance quality factor.

The method 600 includes a step 640 of processing the one or more characteristics of the noise signal. When calculating the volume of a bladder using acoustic resonance, noise characteristics such as environmental interference, sensor noise, or signal distortions can affect the accuracy of the measurement. To process these noise characteristics, one or more techniques may be used by the smart device 160 or software embodied thereon. In certain embodiments, the one or more techniques include signal filtering, averaging, or Fourier Transform analysis. Furthermore, filtering methods, such as low-pass or band-pass filters, may be used to isolate one or more resonance frequencies from background noise while averaging multiple measurements reduces random noise effects. Additionally, in certain embodiments, advanced signal processing techniques, such as noise suppression algorithms or and the machine learning algorithms 117, can be utilized to further enhance the signal-to-noise ratio.

The method 600 includes a step 650 of estimating a change in volume of the fluid in the toilet based on the processed information obtained in step 640. It is useful to determine the change in volume of the fluid in the toilet because it corresponds to a volume of elimination of a patient. Tracking the volume of fecal and urine elimination is desirable for monitoring pelvic health because it helps to identify dysfunctions, monitor conditions like incontinence or retention, and guide effective treatment plans.

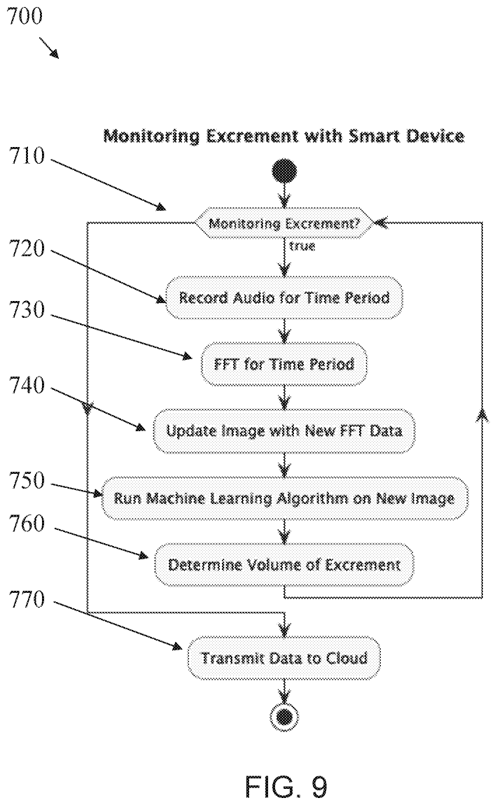

FIG. 9 is a flow chart diagram of a method 700 of monitoring excrement with a smart device, in accordance with one or more embodiments of the present disclosure.

The method 700 includes a step 710 of determining whether the system is currently monitoring excrement from a user. In certain embodiments, the user may be prompted on a user interface of the smart device 160 to provide permission for the system to monitor excrement and collect audio data. The system may then perform a step 720 of recording audio for a time period once permission is received.

The method 700 includes a step 730 of transforming sound data into a Fast Fourier Transform for the time period. Analyzing sound signals in the Fast Fourier Transform space is desirable when measuring the volume of a container (e.g., a bladder) as it fills with fluid because the Fast Fourier Transform converts the time-domain sound signals into their frequency components. As the fluid level rises, the resonant frequencies of the container change due to the changing size of the air cavity. By analyzing the frequency spectrum, particularly shifts in the dominant resonant frequencies (e.g., blue shifts that correspond to higher frequencies), the system can accurately track changes and correlate them to the fluid level within the container.

The method 700 includes a step of updating one or more outputs with new Fast Fourier Transform data that is used to measure changes in frequency that correspond to a changing size of an air cavity as the cavity is filled with fluid. Furthermore, in certain embodiments, the machine learning algorithm(s) may be run on one or more Fast Fourier Transform images 750 to determine volume changes in fluid in a container (or volume changes of excrement in a toilet) that are associated with blue line shifts in the Fast Fourier Transform image(s).

In certain embodiments, one or more of steps 720, 730, 740, 750, and 760 can be repeated to analyze one or more samples of a user or to provide a more accurate analysis of a sample.

The method 700 may include a step 770 of transmitting data to a memory device 770 (e.g., a cloud storage device or any other form of memory as illustrated and described above).

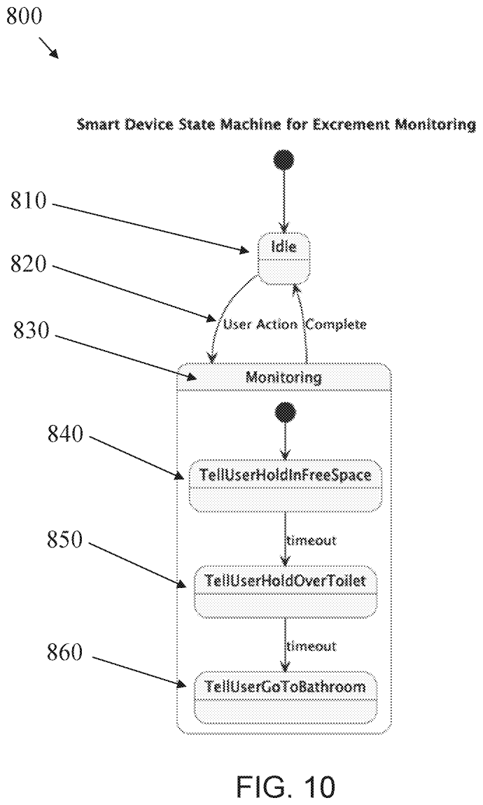

FIG. 10 is a flow chart diagram 800 of another method for monitoring excrement with a smart device, in accordance with one or more embodiments of the present disclosure.

The method 800 may include a step 810 of remaining idle until a user action is completed 820. This may include, by non-limiting example, remaining idle until a user provides permission to record audio during an elimination event or if the user requests the system to take some other form of action.

The method 800 may then monitor the user during the elimination event 830. In certain embodiments, the monitoring events may include instructing a user to hold the smart device in a free space 840 (e.g., holding the smart device away from the toilet to record background noise within the user's environment), instructing the user to hold the smart device of the toilet 850 (e.g., to record noise within the toilet during the elimination event), and instructing the user to go to the bathroom (i.e., performing the elimination event). In certain embodiments, the system may record background noise within an environment prior to recording an elimination event to calibrate the system and filter out undesirable frequencies.

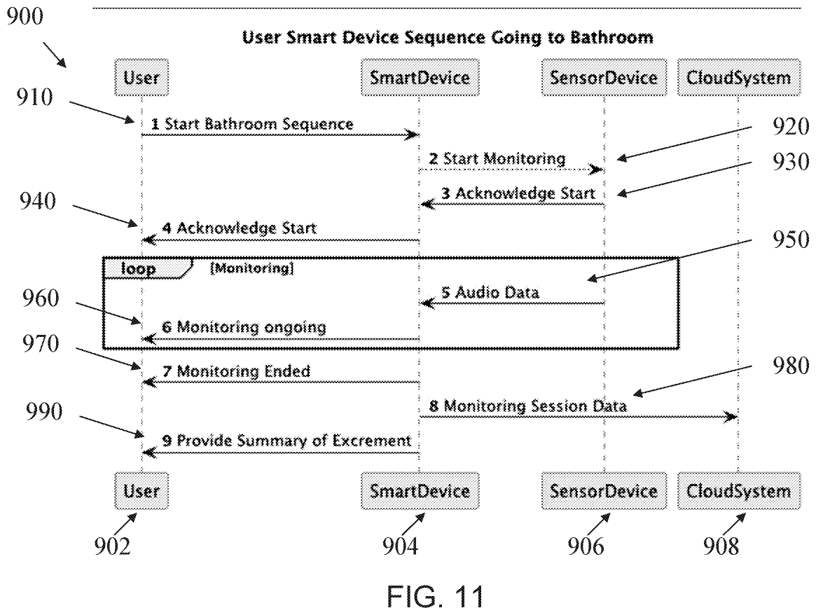

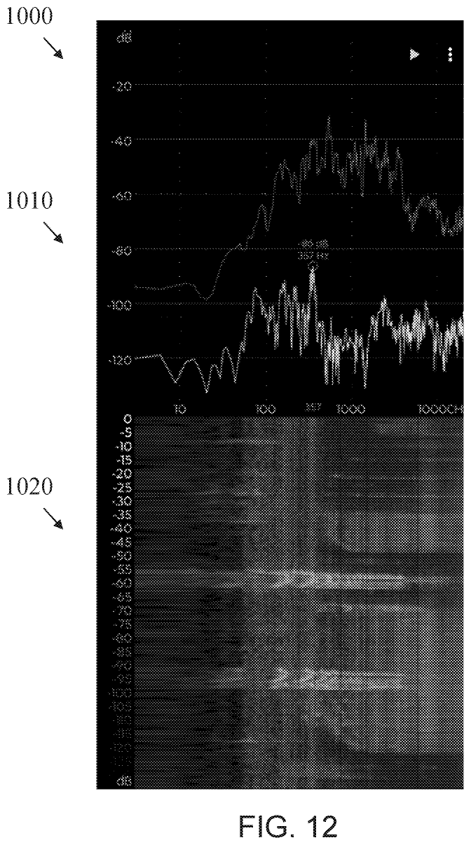

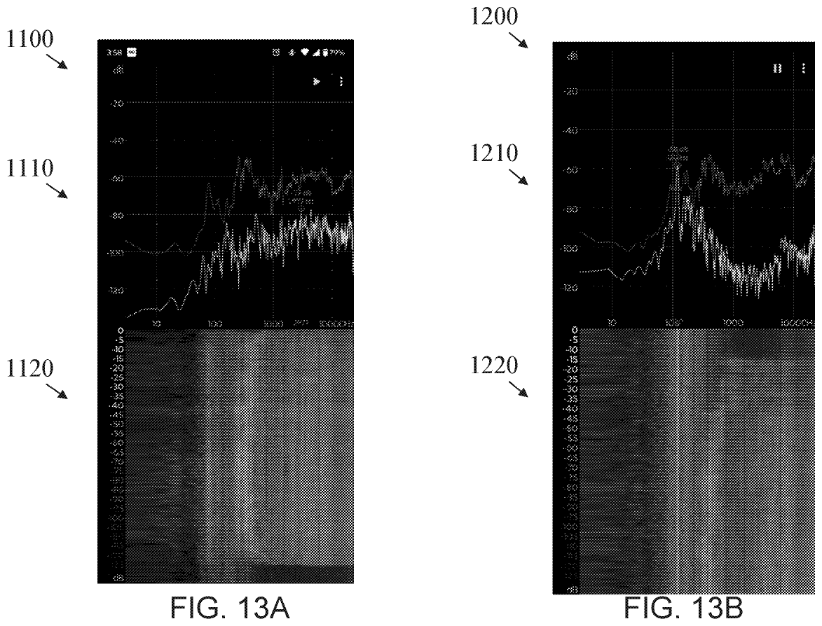

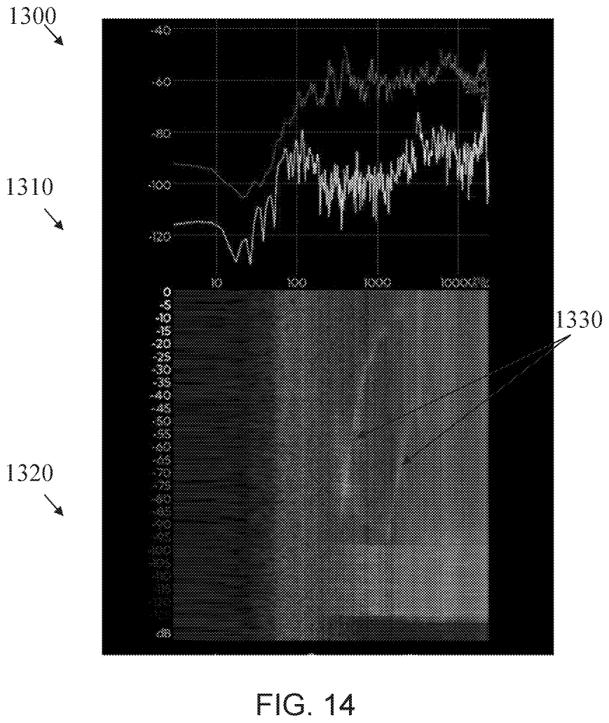

FIG. 11 is a flow diagram of a method for monitoring excrement with a user 902, a smart device 904, a sensor device 906, and a cloud system 908, in accordance with one or more embodiments of the present disclosure. Although a cloud system 908 is included herein, the cloud system 908 may include any usable form of memory as illustrated and described above, including any form of non-volatile memory (e.g., a local hard drive stored on a mobile device).

The method may include a step 910 of starting a bathroom sequence, such as a user entering a bathroom, positioning themselves on or near a toilet, and providing permission for the system to begin monitoring. The system may then start monitoring 920 using a sensor device 906, which then acknowledges the initiation of the monitoring step 930 to the smart device 904. The smart device may then acknowledge the start of the monitoring process back to the user 940.

The method may include a monitoring loop comprising a step of recording audio data using the sensor device 906 and transmitting the audio data to the smart device 950. The smart device 904 may then communicate information relating to ongoing monitoring steps 960 to the user 902. Audio data can be recorded until the elimination event is complete or until the user requests that audio data no longer be recorded.

The method may include a step of ending the monitoring event 970. Session data from an elimination event that is measured by the sensor device 906 can be stored within the smart device 904 and transmitted to the cloud system 908 (or other suitable non-volatile memory) to perform further analysis of the session data (e.g., by using a machine learning algorithm). In certain embodiments, after the session data is analyzed the smart device 904 can provide a summary of the elimination event 990 to a user 902.

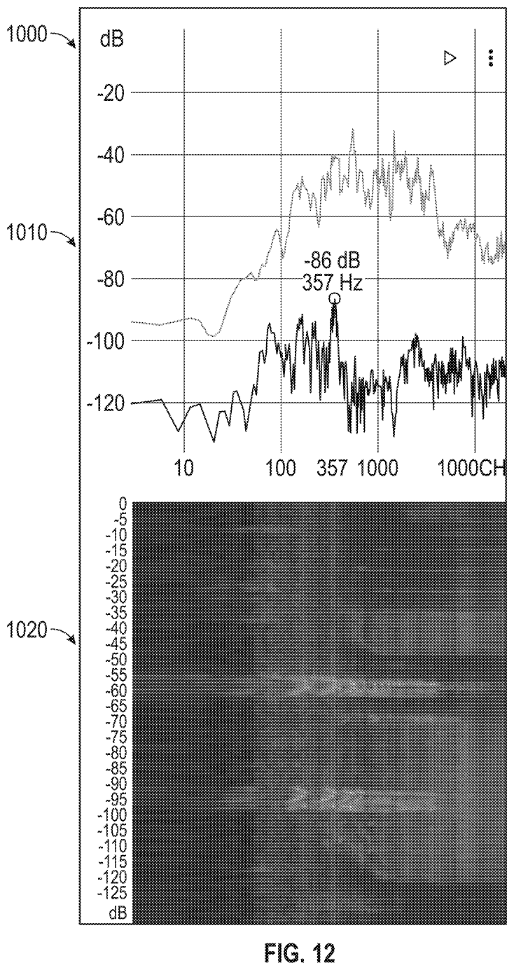

FIG. 12 is graph 1000 of amplitude vs. time 1010 (including a Fast Fourier Transform 1020 illustrating amplitude vs. frequency in the graph below) of an elimination event including background noise, in accordance with one or more embodiments of the present disclosure. The graph 1000 illustrates an increase in amplitude from approximately 30-100 kHz where an elimination event was measured. Furthermore, the Fast Fourier Transform illustrates increases in background noise (e.g., a user's voice being recorded) above 100 kHz.

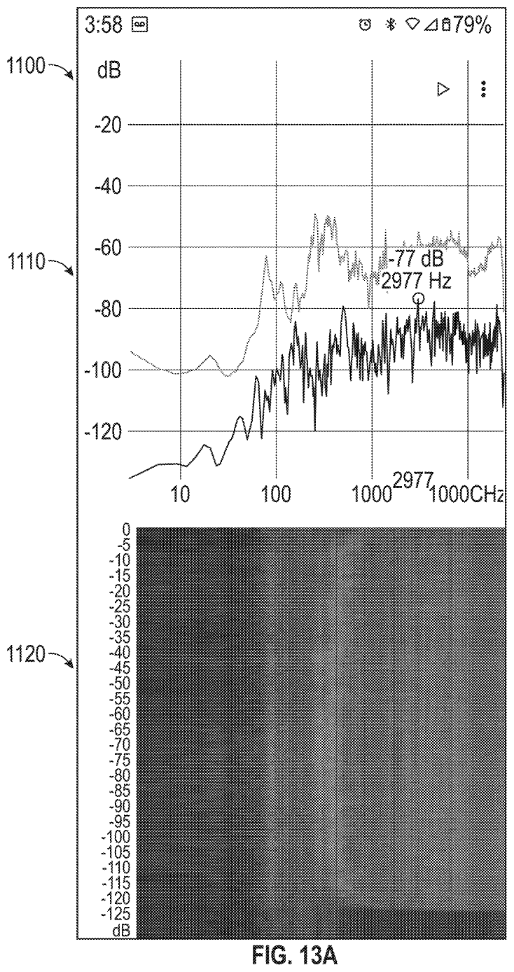

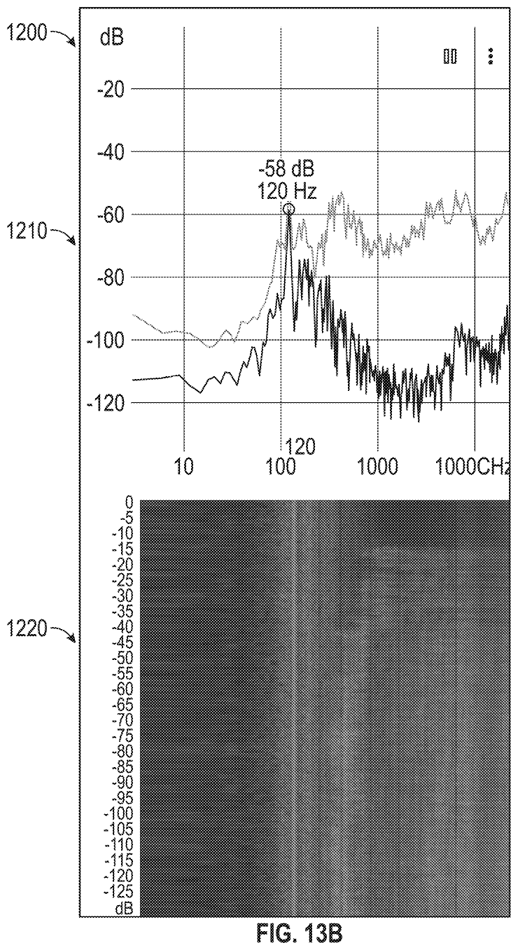

FIG. 13A is graph 1100 of amplitude vs. time 1110 (including a Fast Fourier Transform 1120 illustrating amplitude vs. frequency in the graph below) of an elimination event with a standing male user, in accordance with one or more embodiments of the present disclosure.

FIG. 13B is a second amplitude vs. time (including a Fast Fourier Transform illustrating amplitude vs. frequency in the graph below) of an elimination event with a standing male user, in accordance with one or more embodiments of the present disclosure.

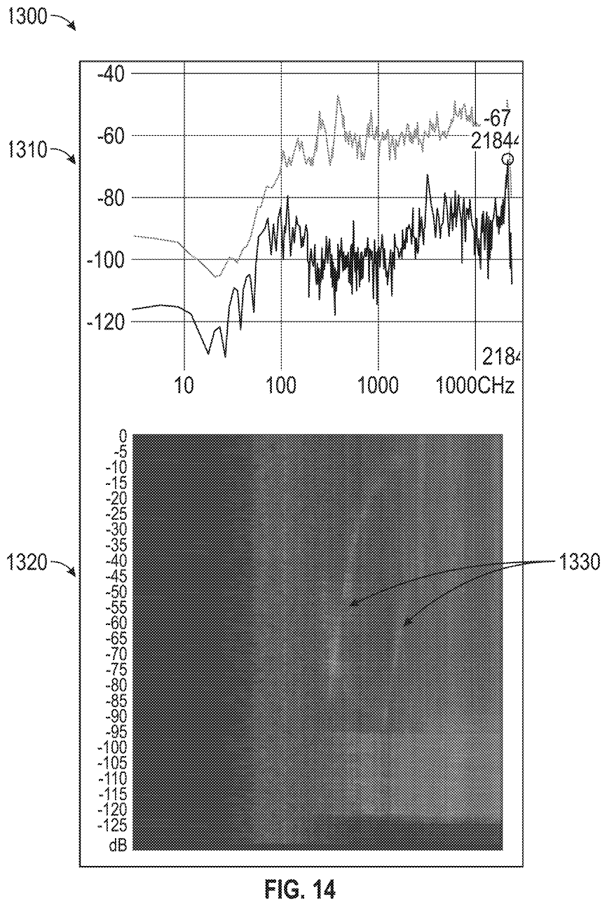

FIG. 14 is a graph 1300 showing two events where sounds associated with filling a container were recorded within an empty space and a blue shift 1330 was observed from approximately 300-1000 Hz and from the time at about the −90 db level, in accordance with one or more embodiments of the present disclosure. As shown in the Fast Fourier Transform 1320, a blue shift 1330 where the slope of the Fast Fourier Transform changing per unit time is the approximately the same for both peaks observed. By tracking the blueshifts in the resonant frequency spectrum (via Fast Fourier Transform), the machine learning algorithm can identify how the container's volume changes over time or under varying conditions. The fact that the blueshifts have approximately the same slope may signal that the system is well-behaved and that the volume changes can be accurately quantified based on the frequency data.

The graph 1300 includes a first plot 1310 illustrating amplitude vs. frequency and a second plot 1320 illustrating a Fast Fourier Transform of amplitude vs. time. Furthermore, the Fast Fourier Transform includes a blue shift 1330 illustrating an increase in frequency as the container fills with liquid, which can be measured and associated with a change in volume of the liquid.

The concept of a “blue shift” in the context of a Fast Fourier Transform (FFT) analysis of sound frequencies relates to the phenomenon where the frequency of a sound increases (shifts to a higher frequency, or shorter wavelength). Measuring blue shifts is desirable when estimating the volume of fluid in a container because the frequency of sound waves within the container is influenced by the physical properties of the system, including the amount of fluid present. As the fluid volume increases, the air cavity decreases in size, leading to a higher natural frequency of the resonating sound waves. This is observed as a “blue shift” in the frequency spectrum after performing an FFT analysis of the sound.

In certain embodiments, the fluid volume can be estimated without direct physical measurement by monitoring the frequency shift. This is useful in scenarios where traditional measurement methods may be impractical, such as in sealed containers or dynamic systems. In some embodiments, the machine learning algorithm can identify relationships between frequency and fluid volume and calibrate the system based on these relationships to allow for precise estimations of volume based on the observed frequency shift.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The aspects of the invention described herein are provided by way of example and are not intended to be limiting to the disclosure in any manner. It should be understood that various modifications, substitutions, and alterations may be made to the embodiments described without departing from the scope and spirit of the invention. The features and elements described herein may be combined in various ways, and additional features may be incorporated as would be understood by those skilled in the art. The disclosure is intended to encompass all such variations, combinations, and equivalents that fall within the scope of the appended claims.

According to a first aspect, the present disclosure includes a system for determining a volume of fluid in a bladder comprising a transducer positioned within the bladder and configured to emit a random noise signal, a receiver positioned within the bladder and configured to receive a response to the random noise signal, and processing circuitry. The processing circuitry may be configured to: generate a first random noise signal when the bladder is substantially empty using a signal transducer, transmit the first broadband random noise signal using a transducer positioned in the bladder, receive a first return signal using a receiver positioned in the bladder, determine a first resonant frequency of the return signal, and determining, based on the resonant frequency of the return signal, the calibrated bladder volume. The processing circuitry may be further configured to determine the volume of fluid in the bladder by: generate a second random noise signal when the bladder contains the fluid, transmit the second random noise signal using the transducer positioned within the bladder, receive a second return signal using the receiver positioned within the bladder, determine a second resonant frequency of the second return signal, and determine a volume of fluid in the bladder by comparing the first resonant frequency and the second resonant frequency. The processing circuitry may be further configured to generate, for output to a user, the volume of fluid in the bladder.

In a second aspect of the system of the first aspect or any other aspect, wherein the receiver comprises a microphone configured to receive one or more of the first random noise signal and the second random noise signal.

In a third aspect of the system of the first aspect or any other aspect, wherein the receiver comprises an accelerometer configured to detect physical vibrations that occur when the first random noise signal or the second random noise signal resonate within the bladder.

In a fourth aspect of the system of the first aspect or any other aspect, wherein the receiver comprises an ultrasound device for measuring the volume of fluid in the bladder.

In a fifth aspect of the system of the first aspect or any other aspect, wherein the receiver comprises a first portion along a surface of the configured to face out of the liquid when the receiver is floating in the liquid and a second portion configured to remain in the liquid when the receiver is floating in the liquid.

In a sixth aspect of the system of the first aspect or any other aspect, wherein the processing circuitry is further configured to determining one or more subsequent fluid volume measurements at one or more defined intervals after measuring the volume of fluid in the bladder.

According to a seventh aspect of the present disclosure, a system for determining a volume of material excreted during an elimination event comprising: a receiver configured to receive a noise signal generated from a patient during the elimination event; and a smart device comprising: a processor; and a computer-readable medium storing instructions which, when executed by the processor, cause the processor to: calibrate the smart device to a toilet used by a patient to estimate an initial volume of fluid within the toilet prior to an elimination; receive the noise signal generated from the patient during elimination; identify one or more characteristics of the noise signal comprising at least one of a noise frequency, a noise amplitude, a duration of sound events, and other acoustic properties; process the one or more characteristics of the noise signal using a machine learning model that is trained to correlate the one or more characteristics with a known volume of fluid in the toilet to estimate a total volume of fluid after elimination; estimate a change in volume by subtracting the initial volume of fluid in the toilet from the total volume of fluid after elimination; and generate, for output to a user, the change in volume.

In an eighth aspect of the system of the seventh aspect or any other aspect, the instructions further cause the processor to request permission from the patient prior to receiving a noise signal generated from the patient during elimination.

In a ninth aspect of the system of the seventh aspect or any other aspect, wherein the instructions further cause the processor to determine, based on the one or more characteristics of the noise signal, whether the elimination included a urination, a defecation, or both.

In a tenth aspect of the system of the seventh aspect or any other aspect, wherein the instructions further cause the processor to transmit the change in the volume to a healthcare provider.

According to an eleventh aspect of the present disclosure, a method for determining a volume of fluid in a bladder comprising: determining a calibrated bladder volume by: generating a first random noise signal when the bladder is substantially empty using a signal transducer, transmitting the first broadband random noise signal using a transducer positioned in the bladder, receiving a first return signal using a receiver positioned in the bladder, determining a first resonant frequency of the return signal, and determining, based on the resonant frequency of the return signal, the calibrated bladder volume. The method may further include determining the volume of fluid in the bladder by: generating a second random noise signal when the bladder contains the fluid, transmitting the second random noise signal using the transducer positioned within the bladder, receiving a second return signal using the receiver positioned within the bladder, determining a second resonant frequency of the second return signal, and determining a volume of fluid in the bladder by comparing the first resonant frequency and the second resonant frequency. The method may further include generating, for output to a user, the volume of fluid in the bladder.

In a twelfth aspect of the system of the eleventh aspect or any other aspect, wherein the first random noise signal and the second random noise signal are a broadband noise signal comprising at least one of white noise, brown noise, and pink noise.

In a thirteenth aspect of the system of the eleventh aspect or any other aspect, wherein the receiver comprises a microphone configured to receive one or more of the first random noise signal and the second random noise signal.

In a fourteenth aspect of the system of the eleventh aspect or any other aspect, wherein the receiver comprises an accelerometer configured to detect physical vibrations that occur when the first random noise signal or the second random noise signal resonate within the bladder.

In a fifteenth aspect of the system of the eleventh aspect or any other aspect, wherein the receiver comprises: a microphone configured to receive at least one of the first random noise signal and the second random noise signal; and an accelerometer configured to detect physical vibrations that occur when the first random noise signal or the second random noise signal resonate within the bladder.

In a sixteenth aspect of the system of the eleventh aspect or any other aspect, further comprising measuring the volume of the fluid in the bladder using an ultrasound device.

In a seventeenth aspect of the system of the eleventh aspect or any other aspect, wherein the receiver comprises a first portion along a surface of the configured to face out of the liquid when the receiver is floating in the liquid and a second portion configured to remain in the liquid when the receiver is floating in the liquid.

In an eighteenth aspect of the system of the seventeenth aspect or any other aspect, wherein the receiver has a center of gravity that is closer to the second portion than the first portion.

In a nineteenth aspect of the system of the eleventh aspect or any other aspect, further comprising determining one or more subsequent fluid volume measurements at one or more defined intervals after measuring the volume of fluid in the bladder.

In a twentieth aspect of the system of the eleventh aspect or any other aspect, further comprising providing a recommendation to the user based on the volume of fluid in the bladder.

In a twenty-first aspect of the system of the first aspect or any other aspect, further comprising taking an action with respect to the user regarding the determined volume of fluid.

In a twenty-second aspect of the system of the seventh aspect or any other aspect, further comprising taking an action with respect to the user regarding the determined volume of fluid.

In a twenty-third aspect of the system of the eleventh aspect or any other aspect, further comprising taking an action with respect to the user regarding the determined volume of fluid.

What is claimed is:

1. A system for determining a volume of fluid in a bladder comprising:
a transducer positioned within the bladder and configured to emit a random noise signal;
a receiver positioned within the bladder and configured to receive a response to the random noise signal; and
a volume measurement device comprising:
a processor; and
a computer-readable medium storing instructions which, when executed by the processor, cause the processor to:
generate a first random noise signal when the bladder is substantially empty using a signal transducer;
transmit the first random noise signal using the transducer positioned in the bladder;
receive a first return signal using a receiver positioned in the bladder;
determine a first resonant frequency of the return signal;
determine, based on the resonant frequency of the return signal, a calibrated bladder volume;
generate a second random noise signal when the bladder contains the fluid;
transmit the second random noise signal using the transducer positioned within the bladder;
receive a second return signal using the receiver positioned within the bladder; and
determine a second resonant frequency of the second return signal;
determine a volume of fluid in the bladder by comparing the first resonant frequency and the second resonant frequency; and
generate, for output to a user, the volume of fluid in the bladder.

2. The system of claim 1, wherein the receiver comprises a microphone configured to receive one or more of the first random noise signal and the second random noise signal.

3. The system of claim 1, wherein the receiver comprises an accelerometer configured to detect physical vibrations that occur when the first random noise signal or the second random noise signal resonate within the bladder.

4. The system of claim 1, wherein the receiver comprises an ultrasound device for measuring the volume of fluid in the bladder.

5. The system of claim 1, wherein the receiver comprises a first portion along a surface of the configured to face out of the liquid when the receiver is floating in the liquid and a second portion configured to remain in the liquid when the receiver is floating in the liquid.

6. The system of claim 1, wherein the processing circuitry is further configured to determining one or more subsequent fluid volume measurements at one or more defined intervals after measuring the volume of fluid in the bladder.

7. A system for determining a volume of material excreted during an elimination event comprising:
a receiver configured to receive a noise signal generated from a patient during the elimination event; and
a smart device comprising:
a processor; and
a computer-readable medium storing instructions which, when executed by the processor, cause the processor to:
receive the noise signal generated from the patient during elimination;
identify one or more characteristics of the noise signal comprising at least one of a noise frequency, a noise amplitude, a duration of sound events, and other acoustic properties;
process the one or more characteristics of the noise signal using a machine learning model that is trained to correlate the one or more characteristics with a known volume of fluid in the toilet to estimate a total volume of fluid after elimination;
estimate a change in volume by subtracting an initial volume of fluid in the toilet from the total volume of fluid after elimination; and
generating, for output to a user, the change in volume.

8. The smart device of claim 7, wherein the instructions further cause the processor to request permission from the patient prior to receiving a noise signal generated from the patient during elimination.

9. The smart device of claim 7, wherein the instructions further cause the processor to determine, based on the one or more characteristics of the noise signal, whether the elimination included a urination, a defecation, or both.

10. The smart device of claim 7, wherein the instructions further cause the processor to transmit the change in the volume to a healthcare provider.

11. A method for determining a volume of fluid in a bladder comprising:
determining a calibrated bladder volume by:
generating a first random noise signal when the bladder is substantially empty using a signal transducer;
transmitting the first broadband random noise signal using a transducer positioned in the bladder;
receiving a first return signal using a receiver positioned in the bladder;
determining a first resonant frequency of the return signal;
determining, based on the resonant frequency of the return signal, the calibrated bladder volume;
determining the volume of fluid in the bladder by:
generating a second random noise signal when the bladder contains the fluid;
transmitting the second random noise signal using the transducer positioned within the bladder;
receiving a second return signal using the receiver positioned within the bladder; and
determining a second resonant frequency of the second return signal;
determining a volume of fluid in the bladder by comparing the first resonant frequency and the second resonant frequency; and
generating, for output to a user, the volume of fluid in the bladder.

12. The method of claim 11, wherein the first random noise signal and the second random noise signal are a broadband noise signal comprising at least one of white noise, brown noise, and pink noise.

13. The method of claim 11, wherein the receiver comprises a microphone configured to receive one or more of the first random noise signal and the second random noise signal.

14. The method of claim 11, wherein the receiver comprises an accelerometer configured to detect physical vibrations that occur when the first random noise signal or the second random noise signal resonate within the bladder.

15. The method of claim 11, wherein the receiver comprises:
a microphone configured to receive at least one of the first random noise signal and the second random noise signal; and
an accelerometer configured to detect physical vibrations that occur when the first random noise signal or the second random noise signal resonate within the bladder.

16. The method of claim 11, further comprising measuring the volume of the fluid in the bladder using an ultrasound transducer.

17. The method of claim 11, wherein the receiver comprises a first portion along a surface of the configured to face out of the liquid when the receiver is floating in the liquid and a second portion configured to remain in the liquid when the receiver is floating in the liquid.

18. The method of claim 17, wherein the receiver has a center of gravity that is closer to the second portion than the first portion.

19. The method of claim 11, further comprising determining one or more subsequent fluid volume measurements at one or more defined intervals after measuring the volume of fluid in the bladder.

20. The method of claim 11, further comprising providing a recommendation to the user based on the volume of fluid in the bladder.
